(19) United States
(12) Patent Application Publication
(10) Pub. No.: US 2012/0215320 A1
(43) Pub. Date: Aug. 23, 2012

(54) METATARSAL HEMIARTHROPLASTY IMPLANT

(76) Inventors: Chris HARBER, Mound, MN (US); Corey WILSON-WIRTH, San Diego, CA (US); Michel HASSLER, St. Ismier (FR); David VANCELETTE, San Diego, CA (US); Nelson OI, (US); Jonathan DELAND, New York, NY (US); Lowell Scott WEIL, SR., Glenview, IL (US); Albert H. BURSTEIN, Reno, NV (US)

(21) Appl. No.: 13/333,760

(22) Filed: Dec. 21, 2011

Related U.S. Application Data
(60) Provisional application No. 61/425,745, filed on Dec. 21, 2010.

(30) Foreign Application Priority Data
Mar. 29, 2011 (FR) 1152587

(51) Int. Cl. A61F 2/42

(52) U.S. Cl. 623/21.19

(57) ABSTRACT

An implant for metatarsal hemiarthroplasty according to embodiments of the present invention includes a resurfacing body configured to at least partially resurface distal and dorsal surfaces of an articular head of a metatarsus, the resurfacing body including a first side and a second side, the first side configured to articulate against a phalange associated with the metatarsus, the first side including a convex surface formed of ceramic, the second side configured to be fixed to the metatarsus, the second side including a concave surface from which protrude at least two immobilization elements, the at least two immobilization elements configured to interface with the articular head.

### CROSS-REFERENCE TO RELATED APPLICATIONS

This application claims the benefit of U.S. Provisional Patent Application Ser. No. 61/425,745, filed on Dec. 21, 2010, and claims foreign priority to French Patent Application No. 1152587, filed on Mar. 29, 2011, both of which are incorporated by reference herein in their entireties for all purposes.

### TECHNICAL FIELD

Embodiments of the present invention relate generally to implants that are adapted to be placed at the articulation between a metatarsus, for example the first metatarsus, and its associated phalange, of a human foot.

### BACKGROUND

In some patients, at the metatarso-phalange level, the articular head of the metatarsus bone is affected, typically by hallux rigidus or by rigid arthritis, which generally cause pain and bother the patient. A possible surgical treatment consists of fusing the metatarso-phalangean articulation, which in turn causes a permanent loss of the joint mobility.

Another approach consists of using an implant only on the phalange, for example a metallic prosthetic component. Another alternate surgical approach is based on inserting a joint implant only on the head of the metatarsus, namely a hemiarthroplasty implant, provided that, after the implant is done, this implant is moving directly against the phalange, and not against another prosthesis. In fact, there are currently several implants for metatarsal hemiarthroplasty on the market—these are fairly large, and involve the metallic body being implanted to replace the anatomical head of the metatarsal bone, with the metatarsal head being either totally resected or significantly cut into. The metallic body is anchored by a central keel, which presents longitudinal and transversal dimensions that allow the mechanical retention of the implant, after having driven the keel deep into the metatarsal bone. Accordingly, the placement of this type of implant results in sacrificing a large volume of metatarsal bone mass, which in turn weakens the bone and complicates any later surgery for fusion of the joint. Moreover, since the prosthesis for the head of the metatarsal bone is typically centered on the longitudinal central axis of the metatarsus, one often sees that, on one hand, the implant interferes with the plantar plate while the patient is walking, which may seriously bother the patient or even damage the sesamoids, and on the other hand, the implant may further lead to dorsal bone spurs of the metatarsus, which are particularly painful for the patient.

### SUMMARY

Embodiments of the present invention include a new implant for metatarsal hemiarthroplasty, which, while being easy to implant, also performs well during use. Accordingly, some embodiments of the present invention include a resurfacing body configured to at least partially resurface distal and dorsal surfaces of an articular head of a metatarsus, the resurfacing body including a first side and a second side, the first side configured to articulate against a phalange associated with the metatarsus, the first side including a convex surface formed of ceramic, the second side configured to be fixed to the metatarsus, the second side including a concave surface from which protrude at least two immobilization elements, the at least two immobilization elements configured to interface with the articular head.

According to some embodiments, ceramic is pyrocarbon. The at least two immobilization elements may protrude from the concave surface along respective directions which are substantially parallel. In some cases, one or more of the at least two immobilization elements which are closest to a plantar side of the implant have a largest protrusion length. According to some embodiments, at least two of the at least two immobilization elements are separated in a median sagittal plane of the resurfacing body. According to some embodiments, at least two of the at least two immobilization elements are each substantially symmetrical with respect to a medial sagittal plane of the resurfacing body. In some cases, the concave surface extends at least partially along a sphere to form a cap. A depth of the cap may be less than 75% of a radius of the sphere, according to embodiments of the present invention.

The second side of the resurfacing body may include a bone contact edge which extends about the concave surface, and which may be formed along a plane, according to embodiments of the present invention.

The convex surface of the resurfacing body may include a central region which has a first radius of curvature taken along a median sagittal cross-sectional plane of the resurfacing body and a second radius of curvature taken along a median horizontal cross-sectional plane of the resurfacing body, wherein the first radius of curvature is smaller than the second radius of curvature. In some cases, the resurfacing body includes a peripheral surface connecting the first side and the second side, wherein the peripheral surface includes a substantially circular contour interrupted by a chord at least on a plantar side of the resurfacing body. According to some embodiments, the resurfacing body is formed in a single piece of ceramic. The ceramic may be pyrocarbon. In some cases, the at least two immobilization elements are at least partially formed of an osteointegration material.

Some embodiments of the present invention minimize the quantity of bone mass that is extracted from the metatarsus for the placement of the implant, while at the same time optimizing the positioning of this implant on the head of the metatarsal bone. Also, the implant body may be used to resurface principally or exclusively the distal side and the dorsal side of the metatarsal head. The plantar side of the metatarsal head is neither resurfaced nor cut into during the bone preparation of the metatarsal head, so as to preserve the plantar plate and the sesamoids and avoid any interference of the implant therewith, including during complete dorsiflexion of the patient's foot. Also, the side of the implant which is to be fixed on the metatarsal head, in covering the distal and dorsal zones prepared beforehand on the metatarsal head, may be concave in order to conserve a maximum amount of bony material present, while lacking a massive central anchor rod or a similar single massive element in favor of at least two distinct protruding elements which are, compared to a single massive element, less embedded within the metatarsus but which, due to their number and/or their relative placement, assure an effective mechanical locking of the implant, notably in rotation and in shear with respect to the metatarsus.

The bulk of the bony material of the metatarsus is also preserved better in avoiding the weakening of the depth of the metatarsus and in preserving the later possibility to fuse metatarso-phalangean articulation, without hurting the mechanical stability of the resurfacing body, according to embodiments of the present invention. The other side of the resurfacing body may be substantially convex, for example in a fashion complementary to the articular surface of the native phalange, as well as cooperating therewith to reproduce the natural kinematics of the original metatarso-phalangean articulation. To restore this articulation as reliably and permanently as possible to the original articulation, the convex articular surface of the implant is formed in ceramic, for example pyrocarbon, according to embodiments of the present invention. In other words, this convex articular surface is formed by a part of the implant body, formed of such a ceramic. As such, the wear on the phalange is significantly limited and/or rendered next to none.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a top perspective view of an implant, according to embodiments of the present invention.

FIG. 2 illustrates a bottom perspective view of the implant of FIG. 1, according to embodiments of the present invention.

FIG. 3 illustrates a top plan view of the implant of FIGS. 1 and 2, according to embodiments of the present invention.

FIG. 4 illustrates a cross-sectional view of the implant of FIGS. 1-3 taken along line IV-IV of FIG. 3, according to embodiments of the present invention.

FIG. 5 illustrates a cross-sectional view of the implant of FIGS. 1-4 taken along line V-V of FIG. 3, according to embodiments of the present invention.

FIG. 6 illustrates a dorsal perspective view of a metatarso-phalangean articulation including the implant of FIGS. 1-5, according to embodiments of the present invention.

FIG. 7 illustrates a cross-sectional view of the metatarso-phalangean articulation taken along the plane IV-IV of FIG. 3, illustrating a placement of the implant of FIGS. 1-5, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

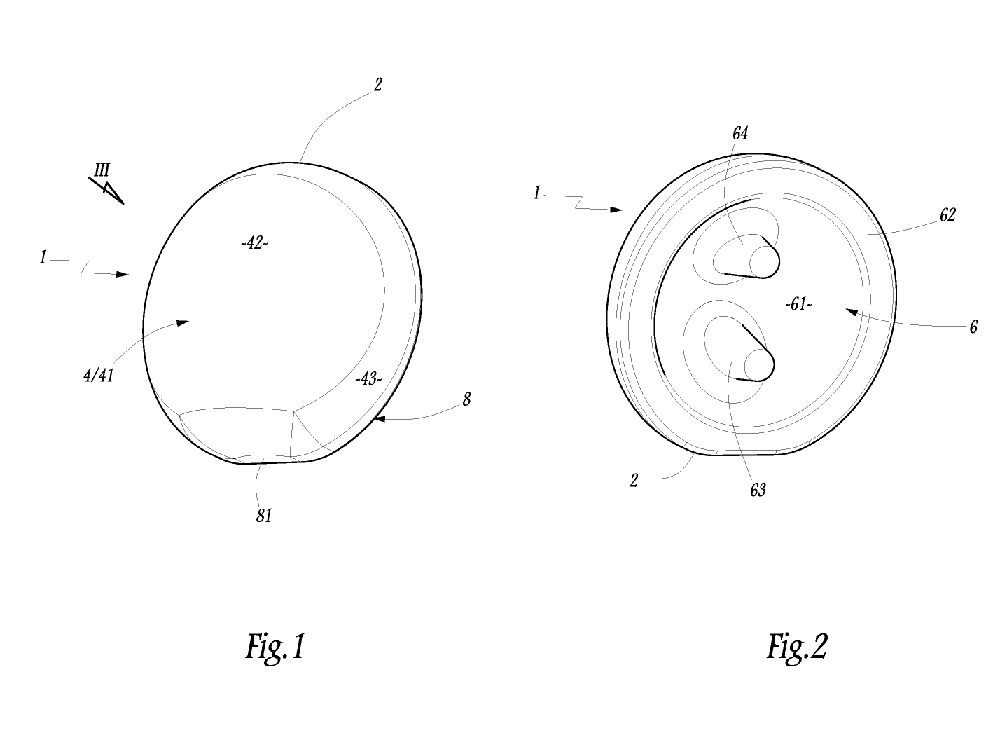

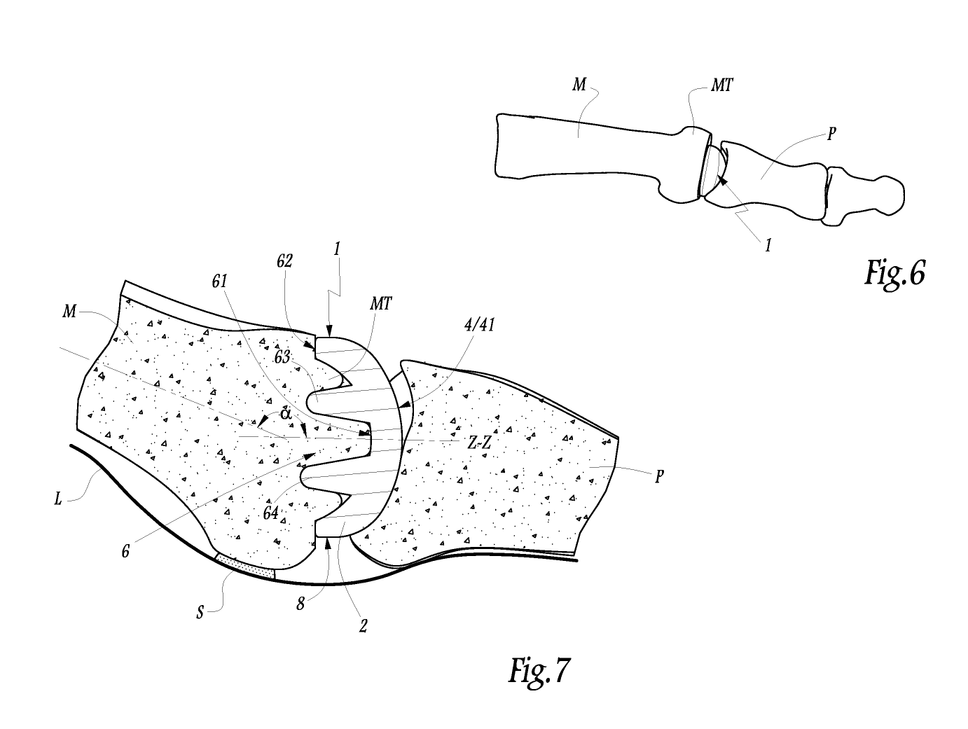

FIGS. 1-5 illustrate a metatarsal hemiarthroplasty implant 1 adapted to be implanted between the metatarsus M, in particular the first metatarsus, and the phalange P associated with the metatarsus, as illustrated in FIGS. 6 and 7, in order to reestablish the metatarso-phalangean articulation between the metatarsus M and phalange P of a foot of a patient.

As used herein, when terms having a usual anatomic sense, such as the term “sagittal,” are used to describe the implant 1, they refer to the implant 1 in its implanted configuration within the metatarso-phalangean articulation.

The implant 1 includes a body 2, which may have an overall shape of a hollow bowl, according to embodiments of the present invention. The body 2 may include two principal opposed faces, 4 and 6, which are separated from each other by the thickness of the body 2 and which, in the embodiment shown in the figures, do not directly connect with each other, but which are connected to each other by a peripheral surface 8 of the body 2, which is cylindrical and of which the generatrix is parallel to a central geometric axis Z-Z. In the implanted configuration of the implant 1, the sides 4 and 6 are respectively facing towards the phalange P and the metatarsus M.

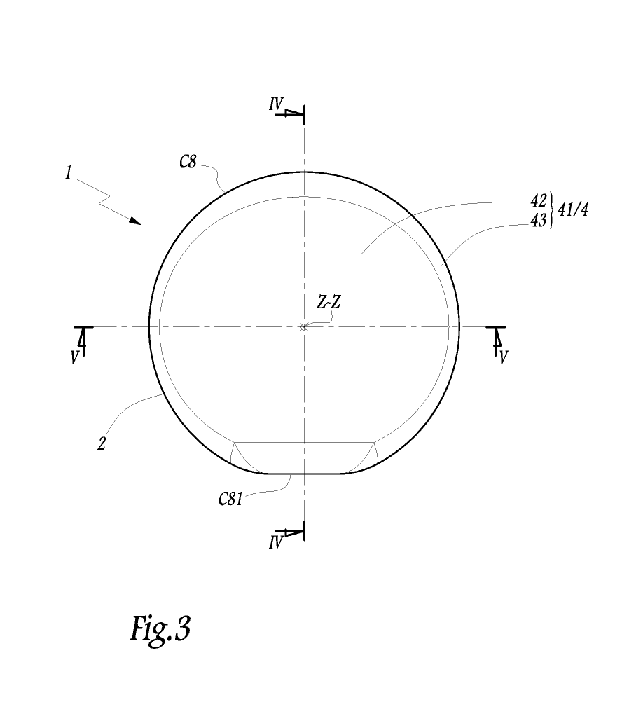

In the embodiment shown in the figures, the peripheral surface 8 of the body 2 is cylindrical with a substantially circular base centered on the axis Z-Z, as shown in FIGS. 3-5. This base need not be perfectly circular. For example, the base may be effectively circular around the whole periphery of the body 2 so as to have a substantially constant radius about axis Z-Z, except on the plantar side of the body 2 (e.g. the portion of body 2 which, in use, is oriented towards the sole of the foot of which the metatarsus M and phalange P are a part. As shown in FIG. 3, the circular contour C8 which the body 2 projects perpendicularly to axis Z-Z is also interrupted by a chord C81 which extends, along a medio-lateral direction, on the plantar side of the contour. The peripheral surface 8 includes, on the plantar side of the body 2, a plate portion 81, which is also visible in FIG. 1, according to embodiments of the present invention. This plate portion 81 may furnish a visual marker for the surgeon's attention, to indicate which surface should face in the plantar direction.

The side 4 of body 2 may extend along or conform to, overall, a portion of a sphere. More precisely, on face 4, the body 2 defines a convex surface 41 which occupies the totality of the face 4.

During use, the convex surface 41 is configured to articulate directly against the phalange P. To this end, the convex surface 41 is formed in ceramic, for example in pyrocarbon, which may also be referred to as pyrolytic carbon. In other words, at least a portion of the surface 41 is formed of such a ceramic. Furthermore, the body 2 may be formed of a single piece, from a ceramic block. This lends to the surface a good biocompatibility, a high mechanical strength, and an elasticity approaching that of bone, while permitting little or no wear on the phalange P.

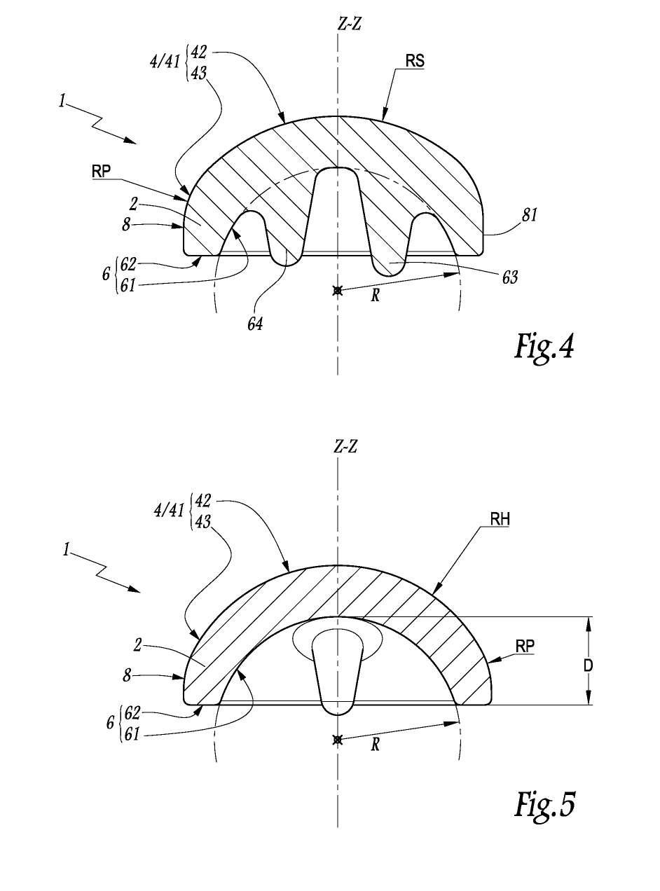

According to some embodiments of the present invention, the convex surface 41 is formed with a non-constant curvature. More precisely, in the central region 42 of the convex surface 41 (e.g. the region which is centered on the axis Z-Z), the surface 41 includes, in the plane of FIG. 4 (e.g. a cross-section taken in a median sagittal plane of the body 2), a radius of curvature RS and, in the plane of FIG. 5 (e.g. a cross-section taken in a median horizontal plane of the body 2), a radius of curvature RH which is larger than the radius of curvature RS, according to embodiments of the present invention.

In this manner, the convex surface 41 includes, at the central region 42, a geometry which is typically complementary to the anatomical articular surface of the phalange P, such that, in the implanted configuration of implant 1, the convex surface 41 articulates against the phalange P with kinematics similar or identical to the natural kinematics of the original, non-prostheticized metatarso-phalangean articulation.

In a similar manner, the peripheral region 43 of the convex surface 41 (e.g. the region of the surface connecting the central region 42 to the peripheral surface 8 of the body), along the entire periphery of the face 4, does not include the same curvature as the central region 42, but a radius of curvature RP shown in FIGS. 4 and 5. The radius of curvature RP is constant along the whole periphery of the region 43, except for the plantar side of the body 2, due to the connection of the central region 42 to the plate portion 81 of the peripheral surface 8, according to embodiments of the present invention. In practice, the radius of curvature RP of the peripheral region 43 is smaller than the radius of curvature RS of the central region 42, and may be less than half of the radius of curvature RS, according to embodiments of the present invention.

According to one non-limiting embodiment, the radius of curvature RS may be between 15 and 30 mm, and the radius of curvature RH may have a value that is about 2 mm greater than the radius of curvature RS, while the radius of curvature RP may include a value between 3 and 5 mm.

The face 6 of the body 2 includes a concave surface 61 which need not occupy the entire face 6, but which may occupy the central region of the face 6, in being surrounded by an edge or border 62 defined by the body 2 all around the concave surface 61, according to embodiments of the present invention.

The concave surface 61 may be spherical in nature, such that the surface 61 corresponds to a cap of a geometric sphere indicated partially in dashed lines in FIGS. 4 and 5. The geometric plane which, by its intersection with this geometric sphere, determines the cap 61, is perpendicular to the axis Z-Z and corresponds substantially to a plane containing the edge 62. Also, the cap 61 is centered on the axis Z-Z, in the sense that the axis Z-Z constitutes an axis of revolution for the shape of the cap 61, according to embodiments of the present invention. The radius of the cap 61 is shown as R in FIGS. 4 and 5. In particular, with respect to the numerical values provided, above, the value R may be at least 2 mm smaller than the radius of curvature RS, according to embodiments of the present invention.

By definition, the cap 61 corresponds to at least a portion of a demi-sphere; in other words, the defining plane of the cap 61, by intersecting the geometric sphere indicated in dashed lines in the figures, does not pass through the center of the sphere. The depth D of the cap 61, in other words the distance along axis Z-Z between such plane and the surface 61, is less than seventy-five percent of the radius R, according to embodiments of the present invention.

Also, as shown in FIGS. 2, 4, and 5, the body 2 is provided, on its face 6, with two distinct elements 63 and 64 which extend as protrusions or projections from the concave surface 61, according to embodiments of the present invention. The elements 63 and 64 protrude from the concave surface 61 along respective directions which are substantially parallel to the axis Z-Z, according to embodiments of the present invention. According to some embodiments of the present invention, the elements 63 and 64 extend from the surface 61 across to the other side of the plane containing the edge 62, according to embodiments of the present invention. The projection length of the element 63, in other words the projection length of the two elements the closes to the plantar side of the body 2, is greater than the projection length of element 64. As shown in FIG. 4, the elements 63 and 64 are separated in the median sagittal plane of the body 2 in a substantially evenly-spaced manner, such that, in the aforesaid median sagittal plane, the distance separating the respective axes of elements 63 and 64 is substantially equal to the distance between each of these axes and the portion of the peripheral surface 8 closest thereto.

Although elements 63, 64 are shown with a particular shape, one of ordinary skill in the art will appreciate that numerous other shapes and configurations may be employed for the elements 63, 64. As shown in the figures, each element 63, 64 is shown with a thimble- or partial cone-shape centered on an axis parallel to axis Z-Z and diverging in the direction approaching concave surface 61. However, other geometric forms are possible, for example cylindrical shapes with a circular, elliptical, or polygonal base, according to embodiments of the present invention.

At a first operating time, a surgeon accesses the articular zone between the metatarsus M and the phalange P, for example via a dorsal approach. After separating the metatarsus M and phalange P from each other as well as enlarging the interosseus space, the surgeon prepares the metatarsal head MT in a manner conforming to or interfacing with the face 6 of the resurfacing body 2, according to embodiments of the present invention. To accomplish this, the surgeon uses, for example, a reamer which applies pressure on the dorsal and distal faces of the metatarsal head, to remove the surface layer of the head constituting cartilage and bony matter. The thickness of the cartilo-osseous material thus removed is of the order of 2 to 5 mm only, which is sufficient to remove the altered portions of the metatarsus, as well as on the distal side which, until then, articulated with the phalange P, which on its dorsal side could also be the site bone spurs of arthritic origin. The preparation operation for the metatarsal head MT of the metatarsus M, notably the application of the aforementioned reaming, may be guided by a pin previously implanted in the metatarsus M, according to embodiments of the present invention. After the metatarsal head MT is prepared, it presents a surface which is complementary with the concave surface 61 and the border 62 of the posterior face 6 of the body 2, according to embodiments of the present invention.

A surgeon may then place the implant 1 on the metatarsal head MT, in grooming the prepared surface of the head with the body 2. In moving the face 6 of the body 2 closer to the prepared surface of the metatarsal head MT of the metatarsus M, the elements 63 and 64 penetrate into the bony material of the metatarsus M. If necessary, to facilitate such penetration, holes may be predrilled into the prepared metatarsal head surface. As the elements 63, 64 extend parallel with respect to each other, their insertion into the metatarsus M may be conducted with an application of force along the direction of the parallelism of the immobilization elements, for example along axis Z-Z, to facilitate the mechanical engagement of the elements into the bony material of the metatarsus M. In particular, the penetration of elements 63, 64 into this bony material is achieved with impaction applied on the face 4 of the body 2, according to embodiments of the present invention.

In any case, as soon as the surface 61 makes contact with the complementary prepared surface of the head MT, the elements 63 and 64 extend in the bony material of the metatarsus M, mechanically immobilizing as well the body 2 with respect to the metatarsus M, for example in rotation about axis Z-Z and/or in shear or sliding transverse to axis Z-Z. At the same time, the edge 62 presses against a complementary dedicated portion of the prepared surface of the head MT, as shown in FIG. 7, notably after the transmission of forces along axis Z-Z between the implant 1 and the metatarsus M, according to embodiments of the present invention.

Once the implant 1 is positioned on the metatarsus M, the separation imposed between the metatarsus M and the phalange P may be released, such that, under the action of the ligaments (eventually reconstructed by the surgeon), surrounding the articulation, the phalange comes to rest against and cooperate in an articular manner against the convex surface 41 of the body 2 of the implant 1. The surgeon can then reclose the soft tissues around the metatarso-phalangean articulation, according to embodiments of the present invention.

The body 2 of the implant 1 permits a distal and dorsal resurfacing of the metatarsal head MT. This is due to the fact that the body presents only a small thickness (e.g. a small distance separates the convex surface 41 and the concave surface 61), which corresponds to the thickness of the cartilage and bone which has been withdrawn from the metatarsal head M T, during the preparation of the head MT. With respect to this small thickness of the body 2, typically on the order of 2 to 5 mm in the numerical example given above, the dorsal and distal positioning of the body 2 on the metatarsal head MT, aiming to resurface the head MT, avoids any interference of the implant 1 with the sesamoids S and the plantar plate L, as shown in FIG. 7, including during walking movements of the foot, notably during a complete dorsiflexion of the foot, according to embodiments of the present invention.

In the context of preserving the sesamoids S and the plantar plate L, the extent of the posterior face 6 of the body 2 may be limited. In limiting the depth D of the cap 61 to 75% of the radius R, as described above, the extent of the metatarsal head MT which is covered by the implant 1 may be limited. For example, an implant 1 according to embodiments of the present invention may be configured to cover no more than three-fourths of the metatarsal head MT, according to embodiments of the present invention.

Also in the context of preserving the sesamoids S and plantar plate L and the optimization of the extent of the coverage of the metatarsal head MT, the implant 1 may be put in place in a manner such that its axis Z-Z is inclined approximately 165° with respect to the central axis of the metatarsus M, as shown by angle a in FIG. 7, according to embodiments of the present invention.

In addition, the absence of interference between the body 2 and the sesamoids S and the plantar plate L is improved by the plate portion 81 of the peripheral surface 8 which is located on the plantar side of the body 2, according to embodiments of the present invention.

Various configurations and variations of the implant 1 and its method of implantation may be used. For example, the number and/or the positioning of the immobilizing elements on the posterior face 6 of the implant, such as the elements 63 and 64, may be modified. Also, three or four, or at least two such elements may be used to guarantee a satisfactory mechanical immobilization, while also limiting the projection length of such elements with an eye toward preserving the bony material of the metatarsus M. According to one variation (not shown), several of the elements 63, 64 may be disposed in a median sagittal plane of the body 2, two similar elements may be disposed in another plane intersecting axis Z-Z, notably in the median horizontal plane of the body 2. In this case, the two elements may each be substantially symmetrical with respect to a median sagittal plane of the body 2, so as to equilibrate the mechanical immobilization forces of the implant 1 on the metatarsus M. In addition, the element or elements 63 closes to the plantar side of the body 2 may be longer than the other immobilizing elements. Effectively, such elements 63 are engaged in a thicker zone of the metatarsal head MT, which permits the implant 1 to benefit from better mechanical stabilization by creating a larger lever arm to resist forces during dorsiflexion of the big toe. Moreover, the longest elements 63 provide the surgeon a supplemental point of reference (e.g. visual point of reference) to facilitate the placement and/or orientation of the implant.

According to another embodiment of the present invention, all or a part of the face 6 of the body 2 may be formed from a different material (e.g. different from ceramic and/or pyrocarbon). For example, the projecting elements, such as elements 63, 64, may be at least partially formed of a material favoring osteointegration, with the aim of reinforcing the mechanical fixation of the elements in the bony material constituting the metatarsus M. Independently of the nature of the material utilized to form the face 6 of body 2, another variation (not shown) involves immobilization elements 63, 64 which are not integrally formed with the concave surface 61, but which may be selectively added by the surgeon. This may be done, for example, preoperatively and/or preoperatively. The concave surface 61 may include several receptacles, for example threaded receptacles, to receive the immobilization elements 63, 64, and the surgeon may customize the placement of such elements 63, 64. The elements 63, 64 may be assembled to the implant 1 to fix them, for example permanently, by screwing them in and/or by pressure fitting them to the receptacles in the concave surface 61, according to embodiments of the present invention.

According to some embodiments, the resurfacing body 2 is symmetrical about its median sagittal plane, and may therefore by used with either a left metatarsus or a right metatarsus, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. An implant for metatarsal hemiarthroplasty, the implant comprising a resurfacing body configured to at least partially resurface distal and dorsal surfaces of an articular head of a metatarsus, the resurfacing body comprising a first side and a second side, the first side configured to articulate against a phalange associated with the metatarsus, the first side comprising a convex surface formed of ceramic, the second side configured to be fixed to the metatarsus, the second side comprising a concave surface from which protrude at least two immobilization elements, the at least two immobilization elements configured to interface with the articular head.

2. The implant of claim 1, wherein the ceramic is pyrocarbon.

3. The implant of claim 1, wherein the at least two immobilization elements protrude from the concave surface along respective directions which are substantially parallel.

4. The implant of claim 1, wherein one or more of the at least two immobilization elements which are closest to a plantar side of the implant have a largest protrusion length.

5. The implant of claim 1, wherein at least two of the at least two immobilization elements are separated in a median sagittal plane of the resurfacing body.

6. The implant of claim 1, wherein at least two of the at least two immobilization elements are each substantially symmetrical with respect to a medial sagittal plane of the resurfacing body.

7. The implant of claim 1, wherein the concave surface extends at least partially along a sphere to form a cap.

8. The implant of claim 7, wherein a depth of the cap is less than 75% of a radius of the sphere.

9. The implant of claim 1, wherein the second side comprises a bone contact edge which extends about the concave surface.

10. The implant of claim 9, wherein the bone contact edge is formed along a plane.

11. The implant of claim 1, wherein the convex surface includes a central region which has a first radius of curvature taken along a median sagittal cross-sectional plane of the resurfacing body and a second radius of curvature taken along a median horizontal cross-sectional plane of the resurfacing body, and wherein the first radius of curvature is smaller than the second radius of curvature.

12. The implant of claim 1, wherein the resurfacing body comprises a peripheral surface connecting the first side and the second side, wherein the peripheral surface includes a substantially circular contour interrupted by a chord at least on a plantar side of the resurfacing body.

13. The implant of claim 1, wherein the resurfacing body is formed in a single piece of ceramic.

14. The implant of claim 13, wherein the ceramic is pyrocarbon.

15. The implant of claim 1, wherein the at least two immobilization elements are at least partially formed of an osteointegration material.
